(19) United States
(12) Patent Application Publication
(10) Pub. No.: US 2026/0015575 A1
(43) Pub. Date: Jan. 15, 2026

(54) METHODS OF AUTOMATED EMBRYOID BODY EMBEDDING IN HYDROGEL USING SEPARATION WELL MICROPLATE

(71) Applicant: Molecular Devices (Austria) GmbH, Puch bei Hallein (AT)

(72) Inventors: Josef Atzler, Hallein (AT); Sara Sofia Deville, Salzburg (AT)

(21) Appl. No.: 19/099,416

(22) PCT Filed: Jul. 20, 2023
(86) PCT No.: PCT/IB2023/057431

§ 371 Date: Jan. 28, 2025

Related U.S. Application Data
(60) Provisional application No. 63/393,567, filed on Jul. 29, 2022.

(51) Int. Cl. C12N 5/00; C12N 5/073

(52) U.S. Cl. CPC C12N 5/0012; C12N 5/0603; C12N 2533/54

(57) ABSTRACT

Methods are provided for automated embedding of embryoid bodies in a hydrogel, media exchange, culturing organoids, and monitoring the development of organoids such as brain organoids. Methods for automated testing of compounds and toxicity effects are also provided.

### CROSS-REFERENCE TO RELATED APPLICATIONS

This application is a Section 371 National Stage Application of International Application No. PCT/IB2023/057431, filed on Jul. 20, 2023, entitled “METHODS OF AUTOMATED EMBRYOID BODY EMBEDDING IN HYDROGEL USING SEPARATION WELL MICROPLATE,” which claims the benefit of and priority to U.S. Provisional Application No. 63/393,567, filed Jul. 29, 2022, the entire contents of which are incorporated herein by reference.

### BACKGROUND

Methods of embedding embryoid bodies in hydrogels for culturing of brain organoids have been difficult to automate. Organoids must be transported with media, but too much media may liquify the hydrogel (e.g., Matrigel) used to encapsulate the embryoid bodies and organoids.

Lancaster & Knoblich (Nat Protoc. 2014 October; 9(10):2329-2340) describe methods for generation of cerebral organoids from human pluripotent stem cells (hPSCs). A labor-intensive process of transferring properly differentiated embryoid bodies (EBs) to Matrigel droplets is described requiring manual preparation of dimpled parafilm substrate for generation of Matrigel droplets. Delicate yet rapid handling of the EBs is required to avoid damage to the aggregate or premature polymerization of the Matrigel.

Improved, automated methods of embedding embryoid bodies in hydrogels for culturing of brain organoids are desirable.

### SUMMARY

Automated methods of embryoid body (EB) embedding in a hydrogel, culturing brain organoids, and methods for automated brain organoid screening are provided.

A method of culturing brain organoids is provided comprising embedding an embryoid body in a liquid hydrogel to encapsulate the embryoid body. The use of a separation well microplate allows for delicate, yet efficient, automatable embedding of the embryoid bodies in a hydrogel. The method may include solidifying the hydrogel encapsulating the embryoid body and suspending the solidified hydrogel encapsulating the embryoid body in a media within a separation well microplate. Methods are provided for shaping the liquid hydrogel encapsulating the embryoid body to become closer to a spherical shape, solidifying of the hydrogel encapsulating the EB, and suspending the hydrogel embedded EBs in a media. Methods are provided for transportation of hydrogel embedded EBs into a bioreactor, feeding and differentiation of EBs into brain organoids, and testing of brain organoids.

A method for culturing of organoids is provided, the method comprising adding an embryoid body into a first media within a primary well of a separation well microplate comprising a plurality of well units, each well unit comprising a primary well, a secondary well, and one or more microchannels connecting the primary well to the secondary well; removing the first media from the well unit via the secondary well; adding a liquid hydrogel to encapsulate the embryoid body; incubating the plate over a first period of time to solidify the hydrogel encapsulating the embryoid body; and adding a second media to the secondary well to detach the solidified hydrogel encapsulating the embryoid body from the plate and suspend in the second media.

In some examples, the adding of the embryoid body into the first media within the primary well comprises automated pipetting.

In some examples, the method for culturing of organoids further comprises tilting the separation well microplate to move the embryoid body to a corner of the main well prior to removing the first media, optionally wherein the tilting comprises automated tilting of the separation well microplate.

The tilting of the separation well microplate may comprise moving the embryoid body toward the microchannels, optionally wherein the tilting comprises tilting the plate to an angle of from 1 to 60 degrees, 5 to 45 degrees, 5 to 35 degrees, 5 to 25 degrees, 5 to 15 degrees, 7 to 12 degrees, or about 10 degrees from a horizontal position.

The tilting of the separation well microplate may comprise moving the embryoid body away from the microchannels, optionally wherein the tilting comprises tilting the plate to an angle of from 1 to 60 degrees, 5 to 15 degrees, 7 to 12 degrees, or about 10 degrees from a horizontal position.

In some examples, the method for culturing of organoids further comprises returning the microplate to a horizontal position before removing the second media.

In some examples, the adding of the liquid hydrogel to encapsulate the embryoid body comprises image guided pipetting of the liquid hydrogel, optionally wherein the image guided pipetting is automated image guided pipetting comprising delivery of the liquid hydrogel on top of at least a portion of the embryoid body.

In some examples, the method for culturing of organoids further comprises cooling the microplate before adding the liquid hydrogel, optionally wherein the cooling is automated cooling to a temperature below room temperature, or within a temperature range of 4 to 8 deg C.

In some examples, the method for culturing of organoids further comprises heating the microplate to incubation temperature before adding the liquid hydrogel, optionally wherein the heating is automated heating.

In some examples, the method for culturing of organoids comprises further tilting the

microplate to move the solidified hydrogel encapsulating the embryoid body away from the microchannels after adding the second media, optionally wherein the further tilting comprises automated further tilting.

In some examples, the incubating to solidify the hydrogel occurs over a first period of time in a range selected from the group consisting of from about 5 minutes to about 90 minutes, about 10 minutes to about 60 minutes, about 20 minutes to about 40 minutes, and about 30 minutes, optionally wherein the microplate is held at room temperature for a second period of time prior to the incubating step.

In some examples, the method for culturing of organoids further comprises exchanging the second media in the well unit via the secondary well periodically to feed the embryoid body.

In some examples, the method for culturing of organoids further comprises introducing a biocompatible oil into the primary well after adding the liquid hydrogel to encapsulate the embryoid body and before the incubating, optionally wherein the introducing comprises automated pipetting of the biocompatible oil into the primary well.

In some examples, the introducing comprises positioning a pipette tip inside the hydrogel and aspirating the hydrogel encapsulating the embryoid body and the oil into the pipette tip, optionally wherein the positioning is image guided positioning. The aspirating may be performed slowly over a period of at least 0.3 seconds, at least 0.5 seconds, at least 0.7 seconds, or at least 1 second.

In some examples, the method for culturing of organoids further comprises pushing the oil and the hydrogel encapsulating the embryoid body from the pipette tip back into the primary well. The pushing may be performed slowly over a period of at least 0.3 seconds, at least 0.5 seconds, at least 0.7 seconds, or at least 1 second.

In some examples, the introducing of the biocompatible oil to the primary well causes the hydrogel encapsulating the embryoid body to become closer to a spherical shape.

In some examples, the method for culturing of organoids further comprises withdrawing the biocompatible oil from the primary well via the secondary well after solidifying the hydrogel encapsulating the embryoid body; and
washing the well unit with a wash media prior to adding the second media, optionally wherein the wash media is at room temperature.

In some examples, the method for culturing of organoids further comprises transferring the encapsulated embryoid body embedded in the solidified hydrogel to a bioreactor for maturation.

In some examples, the method for culturing of organoids further comprises transferring the hydrogel encapsulating the embryoid body and the biocompatible oil to an oil pool that is warmed to incubation temperature to solidify the hydrogel encapsulating the embryoid body.

In some examples, the oil pool comprises a stream that transfers the hydrogel encapsulating the embryoid body to a bioreactor optionally comprising a filter.

In some examples, the method for culturing of organoids further comprises emptying the oil from the bioreactor wherein the filter retains the solidified hydrogel encapsulating the embryoid body; washing the bioreactor and retained solidified hydrogel encapsulating the embryoid body; and adding a third media to the bioreactor to feed the washed hydrogel encapsulating the embryoid body.

In some examples, the method for culturing of organoids further comprises, wherein the oil pool stream that transfers the hydrogel encapsulating the embryoid body into the bioreactor meets with a third media; and the method further comprises allowing the mixture of oil and third media to separate into layers; and removing the oil layer through a filter system to retain the solidified hydrogel encapsulating the embryoid body in the third media; and directing the solidified hydrogel encapsulating the embryoid body in the third media to the bioreactor.

In some examples, the method for culturing of organoids is an automated method, wherein the adding of the embryoid body to the first media, the removing of the first media from the well unit, the adding of the liquid hydrogel, and the adding of the second media each comprise automated pipetting.

The organoids may be brain organoids. The organoids may be liver organoids.

In some examples, the embryoid body is a differentiated embryoid body exhibiting germ layer differentiation.

In some examples, the organoids are brain organoids. The brain organoids may be cerebral organoids.

In some examples, the hydrogel is an extracellular matrix. For example, the hydrogel may be selected from the group consisting of a murine Engelbreth-Holm-Swarm (EHS) sarcoma matrix, a collagen type I, fibrin, hyaluronic acid (HA), gelatin methacrylate (GelMA), a decellularized matrix, alginate, silk, nanocellulose, polyethylene glycol (PEG), a self-assembling peptide, a poly(lactic/(co)glycolic) acid, a polycaprolactone, a polyacrylamide, oligo(ethylene glycol)-substituted poly isocyanopeptide, and an ELP (elastin-like protein).

In some examples, the liquid hydrogel is a cold liquid hydrogel, optionally at a temperature below about 10° C., or in a temperature range from 4 to 8° C. In some examples, the biocompatible oil has a density different than water. The biocompatible oil may have a density higher than water. The biocompatible oil having a density higher than water may be a halogenated hydrocarbon, optionally a perfluorinated hydrocarbon. In some examples, the biocompatible oil has a density less than water. The biocompatible oil having a density less than water may be a silicone oil, a mineral oil, or a vegetable oil.

An automated method for culturing of brain organoids is provided comprising adding a differentiated embryoid body into a first media within a primary well of a separation well microplate comprising a plurality of well units, each well unit comprising a primary well, a secondary well, and one or more microchannels connecting the primary well to the secondary well; tilting the separation well microplate to move the embryoid body to a corner of the main well; removing the first media from the well unit via the secondary well; cooling the microplate and adding a liquid hydrogel to encapsulate the embryoid body in the corner of the primary well of the tilted microplate; incubating the plate over a first period of time to solidify the hydrogel encapsulating the embryoid body; and adding a second media to the secondary well to detach the solidified hydrogel encapsulating the embryoid body from the plate and suspend in the second media.

### BRIEF DESCRIPTION OF THE DRAWINGS

FIG. 1A shows a first view of an exemplary single well unit 100 in a separation well microplate useful for horizontal co-culture for cell/organoid feeding. A primary well 112 is separated from a secondary well 115 by at least one microchannel 118. In some embodiments, the at least one microchannel 118 may be open to allow for media exchange between primary well 112 and secondary well 115. Alternatively, the at least one microchannel 118 may comprise a removable barrier to isolate primary and secondary wells from each other.

FIG. 1B shows a second view of an exemplary single well unit 100 in a separation well microplate useful for horizontal co-culture for cell/organoid feeding. A primary well 112 is separated from a secondary well 115 by at least one microchannel 118. Media exchange occurs by gravity flow through the microchannel 118 while the separation well microplate sits on a rocker.

FIG. 1C shows an example of a cross section of a single well unit 100 of a separation well microplate associated with the cell culturing methods according to various embodiments of the present disclosure. A primary well 112 is separated from a secondary well 115 by a shared sidewall comprising at least one microchannel 118 between the primary well 112 and the secondary well 115. The primary well 112 may contain target cells 103 (e.g., brain organoids) embedded in a hydrogel dome 111. The primary well 112 and the secondary well 115 may be at least partially filled with a liquid media 109. The bottom of the well unit 100 comprises a bottom layer sheet 121 comprising a viewing window that is optically transparent to allow for imaging.

FIG. 2 shows a representative schematic of an exemplary automated work flow method for embedding an embryoid body in hydrogel using a separation well microplate. A cross section of a single well unit of a separation well microplate comprising a primary well 212 separated from a secondary well 215 by a shared sidewall comprising at least one microchannel 218 between the primary well 212 and the secondary well 215. At 200, target cells 203 (e.g., embryoid bodies) are added into the primary well 212 containing a first media 209. At 202, the separation well microplate is tilted to move the embryoid body 203 toward the at least one microchannel 218. At 204, the separation well microplate is leveled and the first media 209 is removed via the secondary well 215 via pipette 220. At 206, a liquid hydrogel 211 is added via pipette 220 on top of the embryoid body 203 in the primary well 212 and incubated to solidify the hydrogel 211. At step 208, a second media 213 is added to the secondary well 215 to suspend the embedded embryoid body 203 in hydrogel 209. The hydrogel 211 is in a half moon or teardrop shape. At step 210, the plate is incubated and second media 213 is periodically exchanged via the secondary well 215.

FIG. 3 shows representative images of properly differentiated embryoid bodies developing into brain organoids at Day 0 (left panel), Day 2 (middle panel), and Day 3 (right panel) after automated embedding in a hydrogel. From day 10 after automated stem cell culture began, organoids were embedded in a hydrogel such as Matrigel to support expansion of neuroepithelia. In this case, the previously described workflow was used (FIG. 2) for embedding the EBs in hydrogel. Note the formation of numerous epithelial buds at Day 3, indicating good differentiation after embedding.

FIG. 4 shows a set of drawings representing photomicrographic images of an automated method of embryoid body embedding in a hydrogel using a 96 well separation well microplate and advanced pipetting protocol. Upper left panel (1) shows the embryoid body in media within the main well, the feeding well with media is also shown. Upper middle panel (2) shows an image of the embryoid body at the corner of the main well in media after tilting the separation well microplate. Upper right panel (3) shows the embryoid body at the corner of main well after media removal from main well via feeding well. Lower left panel (4) shows Matrigel added on top of the embryoid body in main well. Lower middle panel (5) shows an image of pipette tip after slowly removing Matrigel and oil, wherein the embryoid body will stay within the Matrigel, which is now becoming a more spherical shape on top of the oil with higher density at the bottom of the pipette tip. Lower right panel (6) shows the embryoid body successfully embedded in the Matrigel.

FIG. 5 shows an exemplary workflow for an automated brain organoid culture and screening method according to the present disclosure. Briefly, over days 0-4, automated stem cell culture begins with induced pluripotent stem cells (iPSC-derived cells) in 2D culture which may be grown until, for example, about 70-80% confluent using exemplary 6 well culture plates in a media such as an hES media. Morphology of stem cell colonies may be monitored by imaging. Colonies may be disrupted, for example, by brief exposure to enzymes (e.g., dispase, trypsin) and a chelator (e.g., EDTA), washed and suspended to obtain a single cell suspension. EBs are fed, and media (is exchanged. Over days 4-8, single cells are transferred to low attachment, U-bottom 96-well plates, fed and media is exchanged periodically. The low attachment plates may be helpful to avoid EB attachment. Resulting differentiated embryoid bodies are transferred to primary well of passaging plate (separation well microplate) at days 8-9 for embedding of the embryoid body in hydrogel using a separation well microplate. The plates are incubated in large feeding incubator with tilting function and monitoring over days 10-50 to form the 3D brain organoids. Development and growth of organoids can be monitored in Matrigel by automated imaging that allows evaluation of organoids size, volume, and complexity. Test compound addition and measurement of effect may be performed after day 50-51, for example, using imaging and 3D analysis for quantitative characterization. In this example workflow, about 42 k compounds (210 k organoids) can be screened within about 6 months. About 800 plates can be accommodated in the tilting incubator used for feeding and monitoring of organoids.

FIG. 6A shows an embryoid body embedded in a spherical Matrigel® hydrogel at the top Fluorinert™ FC-40 biocompatible oil in a pipette tip.

FIG. 6B shows an embryoid body embedded in a spherical GrowDex® cellulose hydrogel at the top Fluorinert™ FC-40 biocompatible oil in a pipette tip.

FIG. 6C shows an embryoid body embedded in a spherical Collagen I (rat) hydrogel at the top Fluorinert™ FC-40 biocompatible oil in a pipette tip. The use of the biocompatible oil results in a more spherical hydrogel in each hydrogel tested.

### DETAILED DESCRIPTION

Automated methods of embryoid body embedding in a hydrogel, organoid culturing, and organoid screening are provided.

Automated methods of preparing 3D cell models representing brain tissues have been developed.

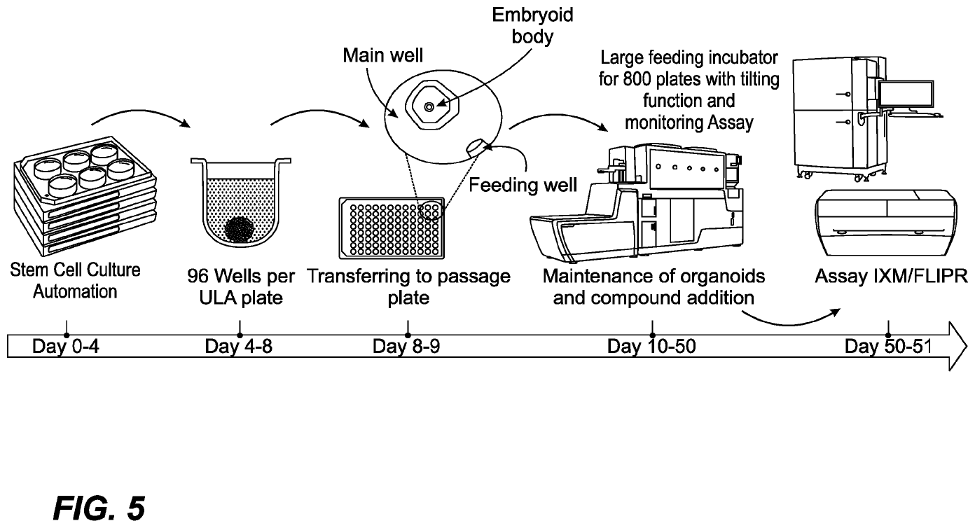

The disclosure provides automated methods of integrated cell culture using a high-content imaging system that allows automated monitoring, maintenance, characterization of organoids, and testing the effects of various compounds. The integrated system includes a confocal imaging system, automated incubator, automated liquid handler, and a collaborative robot. The instruments are controlled by an integrated software that allows for set up of processes. An example of the process to monitor cells in process is shown in FIG. 5, as described herein. Development of EBs and brain organoids may be monitored by imaging. For example, the plates may be moved from the incubator to the imaging system (e.g., ImageXpress Confocal HT.ai, Molecular Devices LLC) for imaging in brightfield than back to the incubator. The process can be scheduled and plates that need to be imaged can be entered in a list to enable easier batch processing. Complex routines that include the liquid handler for media exchanges (feeding) can also be implemented.

Methods are provided for automated embedding of EBs in a hydrogel and media exchange, as well as monitoring the development of brain organoids. In addition, the methods allow for automated testing of compounds and toxicity effects.

### Definitions

The singular forms “a”, “an” and “the” are intended to include the plural forms as well, unless the context clearly indicates otherwise.

The term “and/or” refers to and encompasses any and all possible combinations of one or more of the associated listed items.

The term “about,” when referring to a measurable value such as an amount of a compound, time, temperature, and the like, is meant to encompass variations of 10%, 5%, 1%, 0.5%, or even 0.1% of the specified amount.

Unless otherwise specified, the term “percent” or “%” refers to weight percent.

Unless otherwise specified, the term “density” (d) refers to the mass of a material per unit volume at 25° C.

As used herein, the term “biocompatible oil” refers to an oil that does not adversely affect development, differentiation, or survival of embryoid bodies or organoids when used according to the methods of the present disclosure. The biocompatible oil may be a non-toxic oil when used according to the methods of the present disclosure. The biocompatible oil may have a density different than that of water.

As used herein, the term “composition” may refer to one or more of a compound, mixture, blend, alloy, polymer, and/or copolymer.

The term “room temperature” refers to a temperature in the range of 20 to 25° C., with an average of 23° C.

The term “cold” as in “cold liquid hydrogel” refers to a temperature below room temperature, or within the range of from 0 to 10° C., 1 to 8° C., or about 4° C.

The term “incubate,” “incubating,” or “incubation temperature” refers to about 37° C.

As provide herein, ranges are intended to include, at least, the numbers defining the bounds of the range.

All patents, patent applications and publications referred to herein are incorporated by reference in their entirety.

The term “feeder cells” refers to cells which provide extracellular secretions to help another cell to proliferate, grow, differentiate, and/or maintain identity. Feeder cells may support growth of target cells in culture by contributing a complex mixture of extracellular matrix (ECM) components and growth factors. In some cases, the feeder cells may be unable to divide, i.e., have arrested cell growth. Feeder cell growth may be arrested by, for example, any appropriate methods known in the art. Feeder cell growth may be arrested by chemical fixation, for example, by mitomycin-C or glutaraldehyde chemical fixation. Feeder cell growth may be arrested by physical methods, for example, gamma irradiation, x-ray irradiation, or electric pulses. For example, feeder cells used for co-culture of target cells such as embryonic stem cells (ESCs) may be fibroblasts which may be mitotically inactivated so they remain viable. In some cases, target cells may be grown in the presence of feeder cells capable of dividing. Some live feeder cells (such as human fibroblasts) may also become target cells as in the case of induced pluripotent stem cells (iPSCs) upon reprogramming. Feeder cells may be arrested feeder cells, for example, that are unable to divide. Feeder cell selection may be dependent on target cells. Feeder cells may be, for example, fibroblasts that are not arrested. Feeder cells may be arrested fibroblasts, epithelial cells, mesenchymal cells, muscle cells, stromal cells, spleen cells, or amniocytes. The fibroblasts may be, for example, human dermal fibroblasts, 3T3 fibroblast cells, human fetal fibroblasts, mouse embryonic fibroblasts, and the like. The epithelial cells may be, for example, human adult fallopian tubal epithelial cells, human amniotic epithelial cells, HeLa cells (human cervical cancer carcinoma epithelial cells), and the like. The mesenchymal cells may be adipose-derived mesenchymal stem cells, human bone marrow-derived mesenchymal stem cells, human bone marrow-derived mesenchymal cells, human amniotic mesenchymal stem cells, and the like. The stromal cells may be, for example, human bone marrow stromal cells, or mouse bone marrow stromal cells. The amniocytes may be, for example, human amniocytes or mouse amniocytes.

The term “target cells” refers to cells for automated cell culture applications of the present disclosure, such as embryoid bodies, organoids, tumoroids, spheroids, stem cells, or a production cell line. In some embodiments, the target cells are embryoid bodies, organoids and/or other multi-cellular bodies. The organoids may be brain organoids. The brain organoids may be cerebral organoids. The organoids may be liver organoids. In some embodiments, the target cells may be stem cells. The stem cells may be human induced pluripotent stem cells (hiPSCs). In some embodiments, the target cells may be a production cell line. The target cells may be derived from a target tissue. The target tissue may be a mammalian primary tissue, or an organoid, or tumoroid. The mammalian tissue may be derived from a patient biopsy sample. The target tissue may be derived from target organs such as, e.g., lung, intestine such as small intestine, colon, stomach, pancreas, liver, kidney, skin, bone marrow, blood-brain barrier, brain, heart, and the like.

Organoids, spheroids, tumoroids, and three-dimensional (3D) cell culture models are useful in many applications such as disease modeling and regenerative medicine. 3D cellular models like organoids and spheroids may be useful to better understand complex biology in a physiologically relevant context because cells often retain natural shape and proper spatial orientation, such as in aggregates or spheroids, whereas 2D models of cells grown in a sheet or monolayer may not be as successful. Gene and protein expression of 3D cell culture may more closely mimic gene and protein expression. For example, 3D cell cultures may be useful for drug target identification, lead compound identification, compound optimization, preclinical attesting, solid tumor modeling, genetic disease modeling, drug discovery, precision medicine, organs-on-chips, and bioprinting.

The term “spheroids” refer to three dimensional (3D) multicellular in vitro tissue cultures aggregates composed of one or more cells types that grow and proliferate, and may exhibit enhance physiological responses, but do not undergo differentiation or self-organization. Common cell sources for spheroids are primary tissues or immortalized cell lines. Spheroids may bridge the gap between monolayers and complex organs.

The term “organoids” refers to three dimensional (3D) multicellular in vitro tissue culture aggregates composed of one or more cell types, in which cells spontaneously self-organize into properly differentiated functional cell types and progenitors that resemble their in vivo counterparts. Organoids mimic their corresponding in vivo organs. Organoids can be derived from pluripotent stem cells (PSCs), induced pluripotent stem cells (iPSCs), neonatal tissue stem cells, embryonic stem cells (ESCs), adult stem cells, or primary tissue. Organoid cultures can be crafted to resemble much of the complexity of an organ, therefore are useful for study of disease etiology and treatment. Organoid technology has recently emerged as an essential tool for both fundamental and biomedical research. The organoid cultures may be selected from different types of target organs. The organoids may be, for example, brain organoids, liver organoids, lung organoids, intestine organoids, such as small intestine organoids, colon organoids, stomach organoids, pancreas organoids, kidney organoids, skin organoids, heart organoids, bone marrow, blood-brain barrier, and the like. In the present disclosure, the organoid may be a brain organoid. In some examples, the organoid may be a liver organoid.

The term “brain organoids” refers to 3D tissue models representing one or more regions of the brain. The brain organoid may be, for example, a cerebral organoid, cortical organoid, forebrain organoid, ventral organoid, ventral forebrain organoid, thalamus organoid, hypothalamus organoid, midbrain organoid, hindbrain organoids, cerebellar organoid, medial ganglionic eminence (MGE) organoid, neuromuscular organoid, and the like. Forebrain organoids may include cortical organoids, ventral forebrain organoids, thalamus organoids. Hindbrain organoids may include cerebellar organoids.

The brain organoids may be produced using guided or unguided protocols, for example, as described Kim et al., 2021, iScience 24, 102063, Feb. 19, 2021, which is incorporated herein by reference in its entirety Unguided protocols rely on intrinsic signaling and do not use specific chemicals or inhibitors. For example, cerebral organoids may be self-organized and spontaneously differentiated from hPSCs without extrinsic factors and in the presence of an extracellular matrix (ECM) hydrogel such as Matrigel. The ECM hydrogel may function to support 3D structure and neuroepithelial expansion. Guided methods use extrinsic signaling factors such as signaling activators, or inhibitors to produce region-specific brain organoids. For example, extrinsic signaling factors may include various combinations of SMADi, SHH, insulin, BMP7, MAPK/ERKi, Wnt, FGF2, FGF8, FGF19, SDF1, Noggin, rhDkk1, SB431542, LDN193189, XAV939, WNT3A, purmorphamine, and the like.

Architecture, transcriptome, and function of the brain organoids may be assayed by multiple methods. For example, architecture may be assayed by immunostaining, tissue clearing, or imaging systems. Transcriptome assessments may include qPCR, bulk RNAscq, or scRNAseq methods. Functional assessments may include Ca2+ imaging, microelectrode array (MEA), or patch clamp methods.

When cultured, human induced pluripotent stem cells (hiPSCs) differentiate into various neural cells that mature in time to resemble structures of various brain regions. Cerebral 3D organoids are a developing technology that may be employed for understanding human brain development, neuronal diseases, and can be used for testing the effects of compounds and genetic mutations. The brain organoids may be cerebral organoids. Cerebral organoids may be grown based on the Lancaster and Knoblich protocol (2014). Lancaster and Knoblich, Generation of cerebral organoids from human pluripotent stem cells. Nat Protoc. 2014 October; 9(10):2329-2340, which is incorporated by reference herein in its entirety.

The term “stem cells” refers to undifferentiated cells that have the potential to develop into many different cell types that carry out different functions. Pluripotent stem cells, such as those found in embryos, can give rise to any type of cell such as those in brain, bone, heart, and skin. Some human adult cells can be reprogrammed into embryonic stem cell-like state called induced pluripotent stem cells (iPSCs). The iPSCs may be human iPSCs. Multipotent stem cells, for example, found in adults, or in babies umbilical cords, may develop into the cells that make up the organ system that they originated from. When grown under certain cell culture conditions, pluripotent stem cells can remain undifferentiated. To generate differentiated cells, the chemical composition of the culture media may be changed, the surface of the culture dish may be altered, or the cells may be modified by forcing expression of certain genes.

The term “embryoid bodies” (EBs) refers to three dimensional aggregates of pluripotent stem cells. The EBs may be formed in suspension by pluripotent stem cells (PSC), including embryonic stem cells (ESC) or induced pluripotent stem cells (iPSC). EB differentiation may be used as a common platform to generate specific cell lineages from PSCs. Many EB formation protocols employ components such as fetal bovine serum (FBS) or Knock-Out Serum Replacement (KOSR), or an albumin product. Many human PSCs cannot survive in suspension unless in aggregates or under ROCK inhibitor treatment (e.g., Rock Inhibitor, Tocris).

Using automated systems, methods for culturing three-dimensional brain organoids from human iPSCs are provided comprising automated embedding of embryoid bodies in a hydrogel. In some examples, iPSCs may be grown and dissociated to single cells and allowed to re-aggregate to form embryoid bodies (EBs) in a media such as hES media over days 0-4. Cells may be monitored by imaging, e.g., for EB formation, germ layer differentiation. Over days 4-8, the EBs are transferred to a 96 well U-bottom plate and media is exchanged to a media such as a neural induction media. Feeding and monitoring is performed. After induction of neural ectoderm at about days 8-9, the EBs may be transferred to separation well microplates and embedded in a hydrogel such as Matrigel. A media, such as a differentiation media, may be added. The differentiation media may initially not include vitamin A (retinoic acid). After development of neuroepithelial bud expansion, media exchange may include a differentiation media with vitamin A (retinoic acid). On or about days 10-50 organoid maintenance and optionally compound addition may take place. The separation well microplates may be moved to a feeding incubator with tilting function and monitoring capacity. The feeding incubator may be a large feeding incubator with a capacity of, for example, 10-1000 plates, 20-800 plates or more. At days 50-51, brain organoids may be assayed, for example, using a high throughput cellular screening instrument, such as a FLIPR Penta High-Throughput Cellular Screening System (Molecular Devices LLC). The cellular screening instrument may enable high-throughput screening of the brain organoids for toxicology and lead compound identification. The cellular screening instrument may include an internal plate handler, camera, pipettor heads, heated stage, optics for excitation and emission of fluorescent labels, and computer controller.

Using an automated liquid handling system allowed automated seeding of EBs and addition of hydrogel droplets to embed the EBs, followed by automated media addition and media exchanges. Organoid development was monitored using imaging in transmitted light. Then machine learning-based image analysis allowed detection of organoids and characterization of their size and density. For endpoint measurements, organoids could be stained with fluorescent-labeled antibodies or viability dyes an imaged using the automated confocal imaging system. Advanced image analysis allowed by 3D reconstitution and complex phenotypic evaluation of organoid structures including characterization of organoid size and complexity, cell morphology, and viability, and presence of differentiation markers.

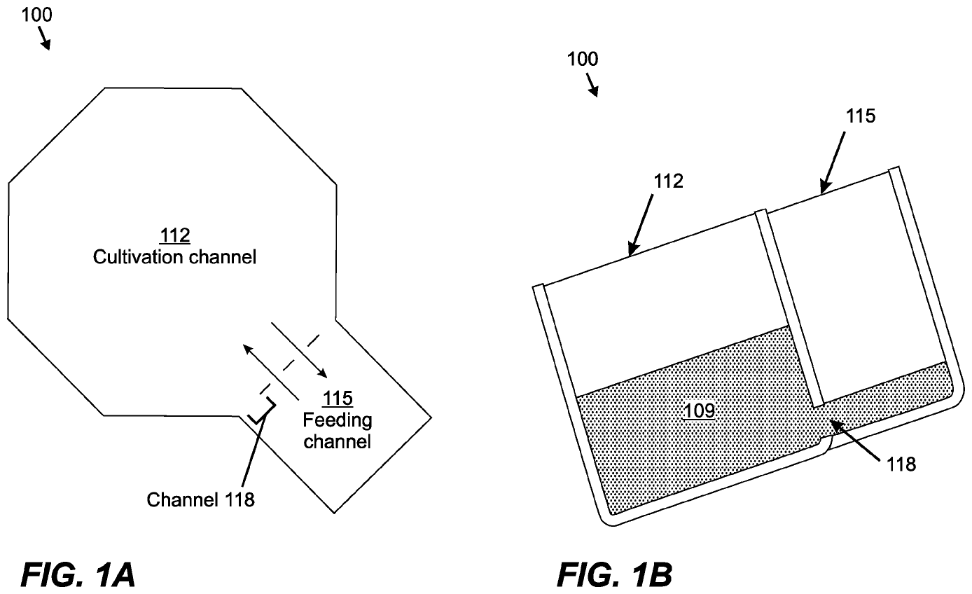

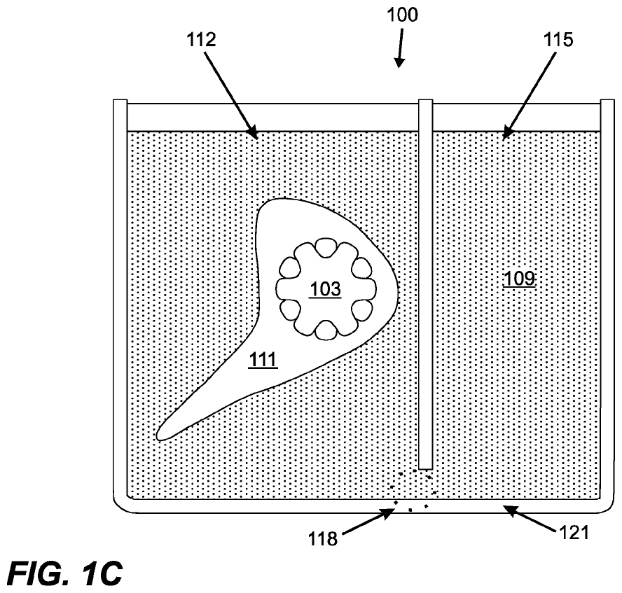

Methods of the disclosure employ a separation well microplate having a well geometry including a primary well and a secondary well connected by at least one channel. For example, the culturing methods of the present disclosure may utilize separation well microplates having a multiplicity of well units each including a primary well and a secondary well connected by at least one channel. For example, each well unit may comprise a well geometry as shown in FIG. 1A comprising primary well that is fluidly connected to a secondary well via at least one channel. Separation well microplates are described in U.S. patent application Ser. No. 17/580,193 entitled “MICROPLATES FOR AUTOMATING ORGANOID CULTIVATION” filed on Oct. 22, 2021, and PCT International Application PCT/IB2021/062356, entitled “MICROPLATE WELLS FOR CELL CULTIVATION” filed on Dec. 27, 2021, each of which is incorporated by reference herein in its entirety.

The size and morphology of the developing brain microtissues may be monitored using automated transmitted light microscopy. An AI-based image analysis (IN Carta software) approach may be used for defining the size, shape, and density of the tissues. For example, microtissues may be analyzed during different phases of development using confocal imaging by the expression of Sox2, TuJ1, and GFAP markers or other markers using, for example, antibody-fluorescent dye conjugates. Organoids may be fixed and stained with Hoechst (blue fluorescent dye used to stain DNA) and Sox2 (radial glia). Analysis of brightfield images may be performed using deep learning-based segmentation (SINAP). Maturation of organoids can be monitored using brightfield imaging and analyzed using IN Carta SINAP. For example, a custom deep-learning model may be trained and then applied to the dataset. The SINAP model allows for segmentation of organoids with different shapes and sizes.

FIG. 1A illustrates one example of a well unit 100 of a separation well microplate that can be used in connection with the methods of the present disclosure. FIG. 1A illustrates an example view of a well unit 100 of a microplate that can be used for growing, culturing, monitoring, and assaying embryoid bodies, fused embryoid bodies, spheroids, organoids, or other multi-cellular bodies in accordance with various embodiments of the present disclosure. For example, the single well unit 100 of a separation well microplate is useful for horizontal co-culture for cell/organoid feeding. A separation well microplate for use in methods according to the disclosure may comprise a plurality of well units, each well unit comprising a primary well 112 that is separated from a secondary well 115 by a shared sidewall, and primary and secondary wells may be fluidly connected by at least one microchannel 118. In some embodiments, the at least one microchannel 118 may comprise a removable barrier to isolate primary and secondary wells from each other. In some embodiments, the at least one microchannel 118 may be open to allow for media exchange between primary well 112 and secondary well 115. The well unit 100 of the separation well microplate includes a primary well 112 which may be fluidly connected to a secondary well 115 by at least one microchannel 118. In some examples, the primary well 112 may be used as a cultivation well and the secondary well 115 may be used as a feeding well. The microchannel 118 may contain a removable barrier. The at least one microchannel 118 may be closed by a removable barrier or may be open. The one or more microchannels 118 between the primary well 112 and secondary well 115 may be closed by a removable barrier, for example, by an air gap, hydrogel seal, or silicone seal. The one or more microchannels 118 between the primary well 112 and secondary well 115 may be closed, for example, by an air gap. The air gap may be formed by, for example, air bubbles.

FIG. 1B illustrates another example well unit 100 of a separation well microplate that can be used in connection with the methods of the present disclosure. In FIG. 1B, the well unit 100 includes a primary well 112 and secondary well 115 that can be fluidly connected by an open at least one microchannel 118. When open, the at least one channel 118 geometry may allow for exchange of media, expressed proteins such as hormones, nutrients, growth factors, waste products, and debris between primary well and secondary wells, while retaining, for example, target cells such as organoids in primary wells 112 and retaining feeder cells in secondary wells 115. The dimensions of the at least one microchannel 118 may prevent the passage of multicellular target cells (e.g., organoids, tumoroids, etc.) through the microchannel 118. In some examples, the at least one microchannel 118 geometry may include a slit configuration having a channel width of 150-500 microns or more, or 200-400 microns, or about 300 microns×10-100 microns, or 10-75 microns high. During the methods of the disclosure, the at least one microchannel 118 between primary and secondary wells may be closed by a removable barrier or may be opened to allow for exchange of media, secreted factors, hormones, growth factors, nutrients, waste products and debris between primary well 112 and secondary well 115, for example, by gravity feed while rocking the separation well microplate. In some examples, the at least one microchannel may be opened by, for example, removing a removable barrier such as eliminating the airgap by using a pipette as a pump, for example, sealing a pipette tip to the secondary well to force liquid flow between the two wells.

FIG. 1C illustrates a further example well unit 100 of a separation well microplate that may be used in connection with the methods of the present disclosure. The well unit 100 includes a primary well section 112, a secondary well section 115, at least one microchannel 118, and an embryoid body 103 embedded in a suspended hydrogel 111 that is disposed in the primary well section 112 of the well unit 100. The primary well 112 and the secondary well 115 contain a media 109. According to various embodiments, the well unit 100 may further comprise a bottom layer sheet 121 disposed on an underside of a well plate body of the microplate. The bottom layer sheet 121 is attached to the underside of the well platebody forming the bottom surface of the well unit 100. In various examples, the bottom layer sheet 121 comprises a viewing window that is optically transparent to allow for imaging of embryoid bodies or organoids, or other cell cultures being cultured in the well unit 100 of the microplate, as can be appreciated. The viewing window can be a window that is suitable for microscopic observation, whether brightfield, phase-contrast, fluorescent, confocal, two-photon, or other microscopic imaging modalities as known in the art. In some examples, the bottom layer sheet 121 may comprise a gas permeable sheet that is configured to increase an oxygen supply for the growing embryoid bodies, and organoids, or other cellular bodies in the well unit 100 of the microplate. The gas permeable sheet can be formed of a material comprising polytetrafluoroethylene (PTFE), PEFP, Polyimide, Polydimethylsiloxane (PDMS), Polycarbonate, and/or other material as can be appreciated. According to various examples, the gas permeable sheet can have a thickness of about 5-70 microns. According to various examples, the gas permeable sheet can comprise a plurality of pores. In other examples, the gas permeable sheet can allow molecules to pass by diffusion. Alternatively, the gas permeable sheet can comprise some other thickness, pore diameter, and pore density.

The secondary well section 115 can be used to grow feeder cells, supply feeding media and/or other nutrients, that can be used to feed the growing cell aggregates positioned in the primary well section 112. The secondary well section 115 can be considered a supply well that comprises the feeding media and/or other nutrients that can be used by the growing cell culture in the primary well section 112. The secondary well section 115 may be sized and shaped to hold fluid that can be exchanged with the primary well section 112 according to various embodiments of the present disclosure.

According to various embodiments, the size and shape of the primary well section 112 and the secondary well section 115 can differ from one another. In some examples, the primary well section 112 is larger (in a dimension, for example diameter, cross-section, or volume) than the secondary well section 115. In other examples, the secondary well section 115 is larger than the primary well section 112. In some examples, the primary well section 112 comprises a shape that differs from a shape of the secondary well section 115.

According to various examples, the well unit of the separation well microplate comprises at least one microchannel 118 that is sized and shaped to prevent objects having dimensions (e.g., diameter, height, width, etc.) of a certain size (e.g., greater than about twenty-five (25) microns (μ)) from passing from one well section to the other well section.

In some examples, the height of the at least one microchannel 118 can be sized between about ten (10) microns up to about one hundred (100) microns, or about 10 microns to about 75 microns, or about ten (10) microns to about twenty-five (25) microns. The at least one microchannel 118 geometry may include a slit configuration having a channel width of 150-500 microns or more, or 200-400 microns, or about 300 microns×10-100 microns, or 10-75 microns high. For example, the height of the at least one microchannel 118 may be such to prevent objects (e.g., embryoid bodies, organoids, organoid fragments, etc.) that are sized at about 25μ or greater that are present in the primary well section 112 will be prevented from migrating to secondary well section 115. The microchannel 118 can be closed by, for example, an airgap, a hydrogel seal, or a silicone seal. The microchannel 118 can be opened by, for example, eliminating the airgap or removing the hydrogel seal or silicone seal.

In some embodiments of the present disclosure, the primary well 112 may be utilized as a cultivation chamber. Initially, the at least one channel 118 may be closed, for example, EBs may be cultivated in the primary wells 112 of the separation well microplate, for example, without mixing. Optionally, feeder cells may be cultivated and allowed to attach to the bottom of secondary wells 115 of the separation well microplate, for example, without mixing. After adherence of feeder cells to secondary wells, the optionally closed at least one channel 118 may be opened. The separation well microplate may be placed on a rocker to allow exchange of media, hormones, growth factors, nutrients, waste products, and debris by gravity feed. In some examples, feeder cells may be cultivated in the secondary chamber 115, allowing EBs embedded in a hydrogel to be cultivated in the primary chamber 112 to be fed through the open at least one channel.

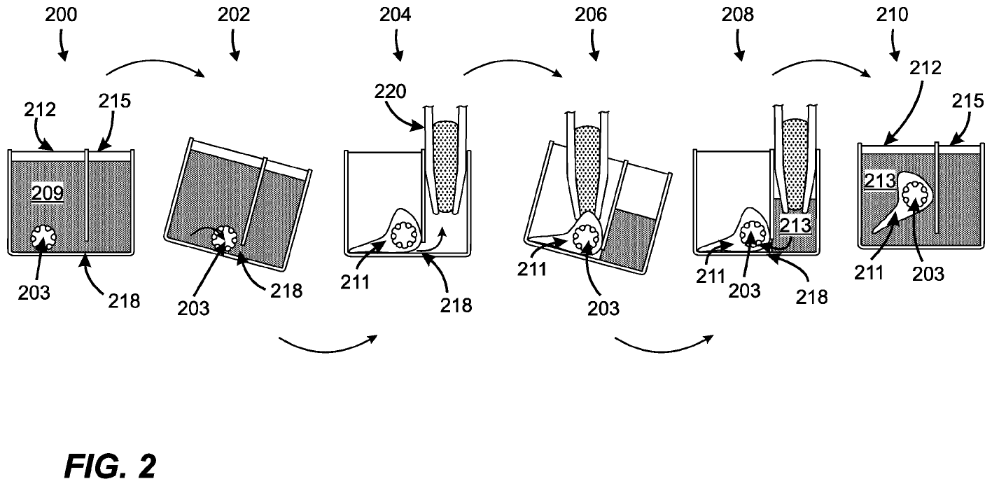

FIG. 2 illustrates a work flow method of embryoid body embedding showing a single well unit of a separation well microplate over time as illustrated in panels 200 to 210. First in single well unit 200, a differentiated embryoid body 203 is pipetted into the primary well 212 of the separation well microplate comprising a first media 209. Secondly in same the single well unit 202, the plate is tilted at an angle (e.g., ˜10 deg) to shift the embryoid body 203 to a corner of the primary well near the channel 218. Thirdly, in the same single well unit 204, the plate is repositioned to a horizontal position and the first media 209 is gently removed from the secondary well 215 (feeding well) via a pipette 220. Fourth, in the same single well unit 206, the plate is angled (˜10 deg) and a liquid hydrogel such as a cold liquid Matrigel 211 is slowly added via pipette 220 to the primary well 212 at the corner near the channel 218 to encapsulate the embryoid body 203. The plate is held for about 5 minutes and allowed to come to room temperature and the plate is moved to the incubator for about 30 minutes to solidify the Matrigel 209. Fifth, in the same single well unit 208, after the Matrigel 209 is solidified, a second media 213 is added from the secondary well 215 (feeding well) to suspend the embedded embryoid body 203 embedded in the Matrigel 211. Sixth, in the same single well unit 210, the plate containing the embedded embryoid body 203 in Matrigel 211 is placed in the incubator and the second media 213 is changed periodically using the secondary well 215 (feeding well) to avoid material loss. The first media and the second media may be different media. The first media and the second media may be the same media. The first media and the second media may differ by one or more, or two or more components. The media may be liquid media at all working temperature ranges (e.g., at cold temperature, room temperature, incubation temperature).

Periodic automated media exchange may be performed, for example, every 24 to 72 hours, 30 to 60 hours, 36 to 54 hours, or at about every 36 hr, about every 48 hr, or about every 54 hours. In some examples, media exchanges may be performed at about every 48 hours.

As shown in FIG. 2, target cells in the form of multicellular bodies 203 (e.g., embryoid bodies) may be embedded in the hydrogel 211 (e.g., Matrigel®) by adding the liquid hydrogel (e.g., cold liquid hydrogel) onto the embryoid bodies in the primary well 212 to encapsulate the embryoid bodies 203. At this stage the hydrogel (e.g., Matrigel®) is typically in a half moon shape. According to various embodiments, a second liquid media 213 is added which contains appropriate growth factors, additives, and supplements to create and/or stimulate growth of the desired multi-cellular body. The liquid media 213 may be added via the secondary well 215 through the open channel 218.

The formation of EBs may employ a common approach to differentiate iPSCs into different cell types.

Prior to automated embedding in a hydrogel, the embryoid bodies may be formed by any appropriate method. Heterogenous methods may include stirred flask culture, liquid suspension culture, Rotary cell culture systems (RCCSS). The homogenous methods may include low adhesion U-bottom multiwell plates, hanging drop culture, indented solid microsurfaces (Aggrewell™), or other methods. The generation of embryoid bodies from iPSCs may comprise an in vitro approach for pluripotency evaluation. The EBs are three-dimensional aggregates of cells which may differentiate to exhibit three developmental germ layers. To assess the pluripotency of hiPSCs to form EBs, a downstream assessment may be performed to demonstrate the ability to form representative germ layers. Assessment may include, for example, histological analysis of EBs, which may include tissue differentiation assessment by investigating tissue organization, cellular morphology, and/or localized protein expression, or by expression of germ layer-specific genes, e.g., to identify the presence of germ-layer-specific gene markers and concomitant loss of pluripotent gene markers.

The methods of the disclosure may employ one or more, two or more, three or more, or four or more different media to produce embryoid bodies and brain organoids. The media may be selected from any appropriate known media in the art. For example, the media may be as described in Lancaster and Knoblich, Generation of cerebral organoids from human pluripotent stem cells. Nat Protoc. 2014 October; 9(10):2329-2340, which is incorporated by reference herein in its entirety.

The media may be any appropriate media known in the art. In some examples, the media may be selected from an hES media, a neural induction media, a cerebral organoid differentiation media without vitamin A (retinoic acid), and a cerebral organoid differentiation media with vitamin A (retinoic acid). The media may be based on a minimal essential media such as DMEM media, Essential Basal Media, Essential 6 Media, commercially available form for example, Invitrogen, or Life technologies.

The media may be an hES media. For example, hES media may be used for generation of embryoid bodies to the point of germ layer differentiation from human PSCs. hES media may comprise DMEM-F12 (Dulbecco's Modified Eagle Media/F12, (Thermo Fisher Scientific), KOSR, FBS, Glutamax (e.g., Gibco™ GlutaMAX™ supplement, L-alanyl-L-glutamine dipeptide substitute for glutamine, Thermo Fisher Scientific), MEM-NEAA (minimum essential media-non-essential amino acids, Thermo Fisher Scientific), and mercaptocthanol, bFGF (fibroblast growth factor-basic, e.g., FGF-basic (143-288), ACROBiosystems) may be added immediately before use. The hES media may be prepared as for example, as described in Lancaster and Knoblich 2014.

The media may be a neural induction media. For example, neural induction media may be used for induction of neural ectoderm in the EBs. Neural induction media may comprise DMEM-F12 with N-2 supplement (e.g., 1% vol/vol) (serum-free supplement based on Bottenstein's N-1 formulation, Thermo Fisher Scientific), Glutamax, (e.g., 1% v/v), MEM-NEAA (e.g., 1% v/v), and heparin (e.g., 1 μg/mL).

The media may be a cerebral organoid differentiation media comprising, for example, DMEM-F12 media, Neurobasal™ media (Thermo Fisher Scientific), N-2 supplement, insulin, MEM-NEAA, penicillin-streptomycin, Gibco™ B27 supplement (optimized serum-free neuronal cell culture supplement, Thermo Fisher Scientific), and/or 2-mercaptoethanol. The differentiation media may be used without or with vitamin A (retinoic acid).

The term “hydrogel” or “hydrogels” refers to an extracellular matrix useful for culturing organoids. The hydrogel may include murine EHS sarcoma matrix, for example, available commercially as Matrigel (Corning), Cultex (Trevigen), Geltrex (Gibco), collagen type I, fibrin, hyaluronic acid (HA), gelatin methacrylate (GelMA), decellularized matrices, or biopolymers such as alginate, silk, nanocellulose; engineered materials such as polyethylene glycol (PEG), self-assembling peptides such as RADA 16/PuraMatrix bQ13, poly(lactic/(co)glycolic) acid, polycaprolactone, polyacrylamide, oligo(ethylene glycol)-substituted polyisocyanopeptides, ELP (elastin-like protein), or combinations of these polymers. The hydrogel may be a Matrigel. Corning Matrigel® matrix is a solubilized basement membrane preparation extracted from the Engelbreth-Holm-Swarm (EHS) mouse sarcoma, a tumor rich in extracellular matrix proteins, including Laminin (a major component), Collagen IV, heparin sulfate proteoglycans, entactin/nidogen, and a number of growth factors. The hydrogel may be Corning® Matrigel® growth factor reduced (GFR) basement membrane mix. The hydrogel may be a collagen hydrogel. The hydrogel may be a collagen type I hydrogel. For example, the hydrogel may be a rat tail collagen type I scaffold (ibibi GmbH, Germany). The collagen scaffold may be 1 to 5 mg/mL, or 2 to 2.5 mg/mL.

The hydrogel may be an animal free hydrogel. The animal free hydrogel may comprise a polysaccharide matrix. The hydrogel may comprise a cellulose matrix. The hydrogel may comprise a cellulose fiber. The cellulose may be a nanofibrillar cellulose. The hydrogel may have a cellulose fiber that is neutral. The hydrogel may have a cellulose fiber charge that is anionic. The hydrogel may be GrowDex® animal free hydrogel (UPM Biomedicals). The hydrogel may be a transparent hydrogel. The hydrogel may comprise a working concentration range of 0.1-1.5 wt %, or 0.2-1.0 wt %. The hydrogel may be a liquid hydrogel. The liquid hydrogel may be a cold liquid hydrogel.

The animal free hydrogel may comprise a self-assembling peptide (SAP). The SAP may be a PuraMatrix® synthetic peptide hydrogel. The SAP may be, for example, a RADA16 peptide or a RLDL-16 peptide. RADA16 is H-(ArgAlaAspAla)₄-OH. Spontaneous and reversible self-assembly of RADA 16 molecules occurs in acidic solutions (pH 2-4, pH˜<3.7) to generate nanofibers. The RADA 16 chemical structure has 16 amino acids as a sequentially repeated 4-amino acid sequence. Containing R (positively charged arginine), A (hydrophobic alanine), and D (negatively charged aspartic acid) residues. The SAPs may be RLDL-16. H-(ArgLeuAspLeu)₄-OH. The extracellular matrix like fibers form viscous and transparent aqueous solutions at low concentrations, e.g., about 0.1 to about 2.5 wt %.

The hydrogel may be a liquid hydrogel under certain conditions. For example, a hydrogel such as Matrigel remains in a liquid state at cold temperatures below room temperature, or from 0 to 10° C., 1 to 8° C., or about 4° C. Upon warming to room temperature, or incubation temperature, the Matrigel solidifies. With respect to self-assembling peptides, under neutral conditions, e.g., RADA16 is mostly in fibrillar form, the fibrils consisting of stacked beta-pleated sheets; under acidic conditions RADA16 fibrils disassemble into monolayers.

In some methods of the present disclosure, a biocompatible oil may be employed. The biocompatible oil may be useful to obtain a more spherical shape of the suspended hydrogel encapsulating the embryoid body. The biocompatible oil may be useful in transporting the suspended the hydrogel encapsulating the embryoid body.

The biocompatible oil will typically have a different density than water. The biocompatible oil may have a density different than that of water. The biocompatible oil may have a density higher or lower than that of water.

The biocompatible oil may have a higher density than water. In some examples, the biocompatible oil may be a halogenated hydrocarbon oil. The halogenated hydrocarbon oil may be a fluorinated hydrocarbon. In some examples, the biocompatible oil may be 1-methoxyheptafluoropropane (e.g., HFE7000 or Novec™ 7000, 3M) or 3-ethoxy-1,1,1,2,3,4,4,5,5,6,6,6-dodecafluoro-2-trifluoromethyl-hexane (e.g., Novec™M 7500, 3M).

The fluorinated hydrocarbon may be a perfluorinated hydrocarbon. The term “perfluorinated hydrocarbon” refers to an organofluorine compound including only carbon-fluorines and C—C bonds, as well as potential functional groups, heteroatoms and associated hydrogen atoms. Common functional groups may include —OH, —CO₂H, —Cl, O, and SO₃H.

The perfluorinated hydrocarbon may have a density at 25° C. (d) greater than 1, 1.2, 1.3, or 1.5, or within a range of from 1.1-2.0, 1.2-1.95, or 1.5-1.95. The perfluorinated hydrocarbon may be a 3M™ Fluorinert™ FC perfluorinated compound (3M Company). Fluorinert FC compounds may include Fluorinert FC-40, perfluoro compounds, C5-C18 (CAS RN 86508-42-1) density at 25° C. (d) 1.85 g/cm³, Fluorinert FC-70 (CAS RN 338-84-1), tris (undecafluoropentyl) amine, d 1.94 g/cm³, Fluorinert FC-770, d 1.79 g/cm³, Fluorinert FC-43 (CAS RN: 311-89-7) perfluorotributylamine, d 1.86, and the like. In some embodiments, the perfluorinated hydrocarbon has a kinematic viscosity of greater than 0.7 cSt, >1.1 cSt, or >2.0 cSt at 25° C.

In some examples, the biocompatible oil has a boiling point greater than 37° C., or greater than 40° C. The biocompatible oil may have a density lower than water. The biocompatible oil having a density lower than water may be, for example, a silicone oil or a mineral oil. Silicone oil is a term used to describe a group of hydrophobic polymeric and monomeric compounds comprising silicone-oxygen bonds and also called organosiloxanes. A silicone oil may be any liquid polymerized siloxane with organic side chains. For example, the silicone oil may be polydimethylsiloxane (d 0.96 g/cm³). The silicone oil may be a cyclosiloxane such as a decamethylcyclopentasiloxane (d 0.96 at 20° C.), or a cyclomethicone D6 (d 0.98). Side chains may include not only methyl, but also phenyl, vinyl, and/or trifluoropropyl groups. Differences in silicone oils may include molecular wt (MW), length of linear chain, type of side chains, chain termination, and size distribution of the chain. Silicone oil with a higher viscosity may have a lower tendency to emulsify. Barca et al., 2014, Silicone oil: different physical properties and clinical applications, BioMed Res Int, article id 502143. The silicone oil may have a viscosity at 25 deg C in a range of from about 5-10,000 cSt, 100 cSt-5,000 cSt, or about 1,000 cSt to about 5,000 cSt. The biocompatible oil may be an oil for tissue culture such as a liquid paraffin oil, white mineral oil (e.g., ORIGIO liquid paraffin for tissue culture, CooperSurgical®). Density of white mineral oil is between 0.8-0.87 g/cm³. In some examples, the biocompatible oil may be a vegetable oil. The vegetable oil may be, for example, an olive oil, corn oil, argan oil, camelina oil, or a mixture thereof. Vegetable oils and mixtures are described in, for example, in Said et al., 2013, Can J Physiol Pharmacol 91: 812-817. The vegetable oil may be commercially available from, for example, Sigma-Aldrich, St. Louis, Missouri, USA

The embodiments described in one aspect of the present disclosure are not limited to the aspect described. The embodiments may also be applied to a different aspect of the disclosure as long as the embodiments do not prevent these aspects of the disclosure from operating for its intended purpose.

### EXAMPLES

### Example 1. Embryoid Body Embedding Using a Separation Well Microplate

In this example, automated embryoid body embedding into a hydrogel was performed. Embryoid bodies were prepared according to the Lancaster and Knoblich protocol (2014), then subjected to automated methods of the present disclosure as outlined in FIG. 5.

As outlined in FIG. 5, generation of EBs from human iPSCs was started in automated stem cell culture plates at day 0 using hES media, media was exchanged, and EB development was monitored for germ layer differentiation via imaging over days 0-4.

Briefly, over days 0-4, automated stem cell culture were started with induced pluripotent stem cells (iPSC-derived cells) grown in 2D culture until about 70-80% confluent using exemplary 6 well culture plates in an hES media. Colonies were disrupted, comprising brief exposure to enzymes and EDTA. (e.g., dispase, washed, then trypsin/EDTA, washed and suspended) to obtain a cell suspension. EBs were fed, and exposed to a first media (e.g., hES media with ROCK inhibitor (1:100) and low bFGF 4/ng/ml). Media was exchanged periodically. Development of cells was monitored by imaging. In some examples, when EBs were ˜350-600 μm in diameter, media was exchanged to hES with no ROCK inhibitor. When EBs were about 500-600 μm in diameter, appearance of the EBs became brighter, with smooth edges, indicating germ layer differentiation.

At about day 4, single cells were transferred to low attachment, U-bottom 96-well plates. Over days 4-8, media was exchanged to neural induction media which was exchanged periodically. Cells were monitored by imaging for induction of neural ectoderm. At about days 8-9, EBs became brighter around the outside indicating neuroectodermal differentiation. Resulting differentiated EBs were transferred via automated pipetting to the primary well of a separation well microplate for embedding in a cold liquid hydrogel (Matrigel).

Automated embedding of EBs was performed as outlined in FIG. 2. At about days 8-9, the differentiated embryoid body (EB) 203 was transferred via pipette arm into the primary well 212 of a separation well microplate 200 containing a first media 209, and EBs were fed via periodic automated media exchange through the secondary well 215 via the microchannel 218 during the differentiation. Once the embryoid body 203 was ready for embedding, the plate 202 was tilted for few seconds till the organoid was situated near the channel 218 (middle wall in the drawing). Afterwards, the plate 204 was re-positioned in a normal flat position and the first media 209 was removed from the feeding well. Once the well was emptied of media, the plate 206 was tilted again and cold Matrigel 211 was added via pipette arm in the corner near the EB 203 and channel 218. After 5 minutes at room temperature, the plate was transferred to the incubator at 37° C. for ˜30 minutes. Once the Matrigel was solidified, a second media 213 (cerebral organoid differentiation media without vit A) was added from the secondary (feeding) well 215 through the channel 218 and in such a way as to resuspend the solidified hydrogel encapsulating the embryoid body in the second media 213 in the separation well microplate 210. The separation well microplates were moved to a large feeding incubator with tilting function, and automated media exchange (cerebral organoid differentiation media with vit A) and monitored via imaging over 10-50 days. The resultant brain organoids were ready for compound testing comprising compound addition and assay at days 50-51, for example, employing image analysis via an ImageXpress Micro Confocal High-Content Imaging System (Molecular Devices LLC) or a high throughput cellular screening instrument, such as a FLIPR Penta High-Throughput Cellular Screening System (Molecular Devices LLC).

The following steps were typically employed for automated embedding of EBs in hydrogel.
1. Pipette embryoid body into the main well of a separation plate and tilt the plate to move the embryoid body to the separation channels.
2. Remove media via the feeding well in a flat position.
3. Tilt the plate and add liquid Matrigel to the main well to encapsulate the embryoid body.
4. Warm up plate to 37° for 30 minutes to solidify the Matrigel.
5. Add media via the feeding channel to detach the embedded embryoid body from the plate bottom.
6. (optional step) Tilt plate to the other direction to move the embryoid body away from the separation channels.
7. (optional step) Add more media to feed the embryoid body.
8. (optional step) Transfer embryoid body to a bioreactor for maturation.

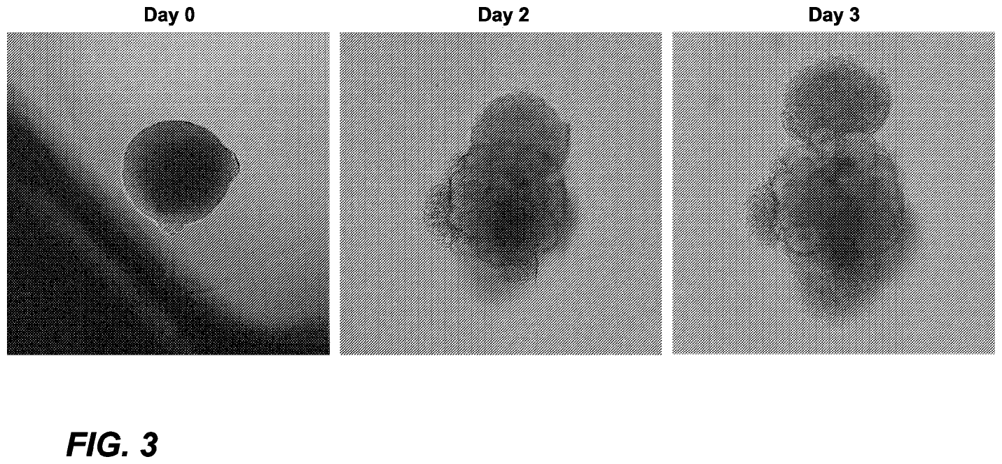

Representative images of proper differentiated Embryoid bodies into Brain organoids are shown in FIG. 3. From day 10, organoids were embedded in Matrigel to support expansion of neuroepithelia. In this case, the previously described workflow was used (FIG. 2). Note the formation of numerous epithelial buds indicating good differentiation at day 3 after embedding.

### Example 2. Embryoid Body Embedding Using Separation Well Microplate and Oil

In this example, automated embryoid body embedding into a hydrogel was performed using a biocompatible oil having a different density than the hydrogel (e.g., Matrigel).

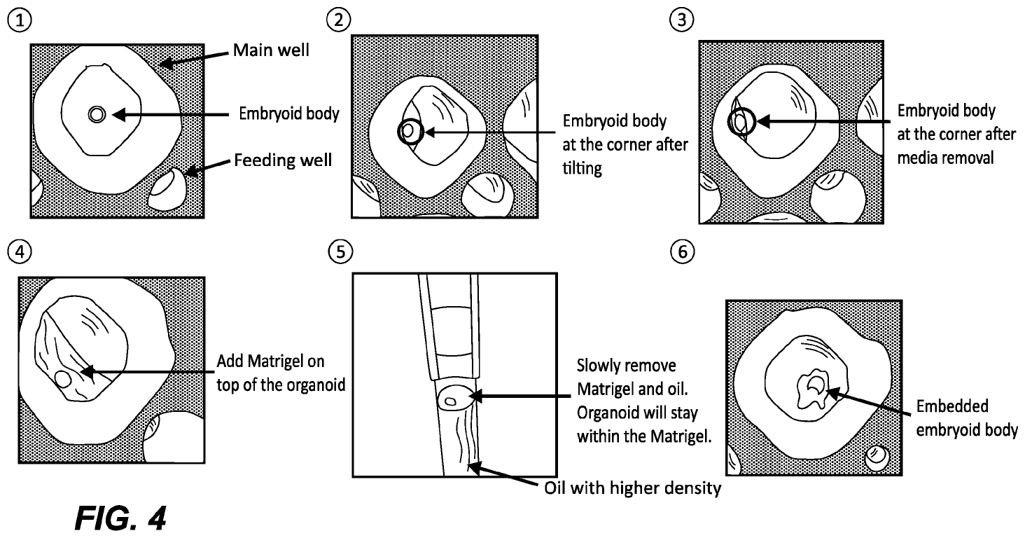

This procedure was similar to that illustrated in FIG. 2, except that after pipetting Matrigel onto the embryoid body to embed the EB in Matrigel, a biocompatible oil having a different density than Matrigel was added to the primary well via pipette to reshape the Matrigel from a half moon shape to a more spherical shape. Two types of biocompatible oil were tested. FluorinertTM FC40 (C5-C18 perfluoro compounds, density at 25° C. of 1.845-1.895 g/mL, b.p. 165° C.) having a density and specific gravity higher than water, and a silicone oil having a density/specific gravity less than that of water. The same pipette tip was positioned inside the Matrigel and used to slowly aspirate the Matrigel and oil. As shown in FIG. 4, the Matrigel was in a separate layer on top of the pipette tip and above the higher density biocompatible oil. Slowly, the oil and Matrigel containing the organoid were pushed back into the well. The plate was then warmed to incubation temperature of 37° C. to solidify the Matrigel. The oil was removed via the secondary well (feeder well), and the wells were washed with media at room temperature. More media was added to feed the embryoid body as shown in FIG. 4 at lower right panel. The separation well microplates were moved to a large feeding incubator with tilting function for feeding and maintenance of organoids and monitored over 10-50 days.

The following steps were typically employed.
1. An embryoid body was placed in the main well of a separation well plate.
2. Tilt the plate to move the embryoid body far away from the separation channel (to the opposite side) to not loose the organoid during media removal.
3. Remove media via the feeding well in a flat position.
4. Tilt the plate and add cold liquid Matrigel to the main well at the corner to encapsulate the embryoid body.
5. Add biocompatible oil to the main well to reshape the liquid hydrogel from a half moon shape to a more spheric shape.
6. Position same pipette tip inside the Matrigel and slowly aspirate the Matrigel and oil.
7. The Matrigel will be then at the top of the tip and the oil due to the density in the lower part (FIG. 4 lower middle panel).
8. Slowly push the oil and Matrigel containing the organoid back to the main well.
9. Warm up plate to 37° for 30 minutes to solidify the Matrigel.
10. Remove the oil via the feeding channel and then wash once with media at room temperature.
11. Finally, add more media to feed the embryoid body.
12. (optional step) Transfer embryoid body to a bioreactor for maturation.

### Example 3. Use of Various Hydrogels for Embryoid Body Embedding

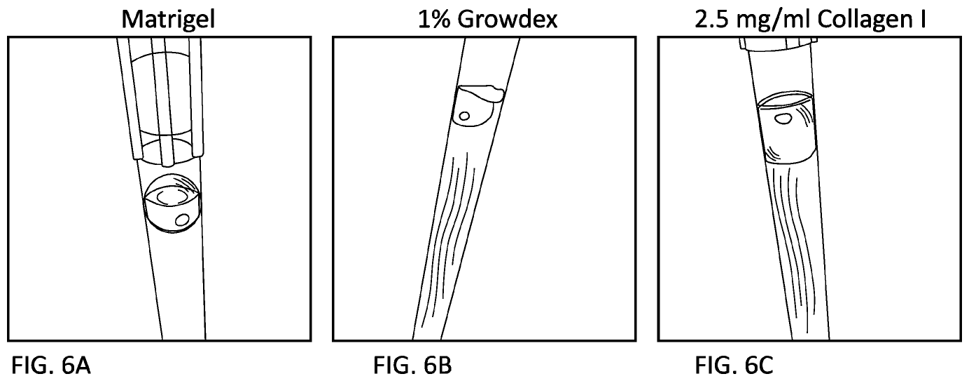

In addition to Matrigel® (Corning), a plant based hydrogel GrowDex® and a Collagen I hydrogel (from rat) were each employed in the methods of the disclosure. Each of the Matrigel, plant based hydrogel, and Collagen I hydrogel behaved in a similar way. Each of the hydrogels had lower density than the biocompatible oil resulting in a spherical shaped hydrogel when exposed to the biocompatible oil. Images of embryoid bodies in the hydrogels above the biocompatible oil in pipette tips are shown in FIGS. 6A-6C. FIG. 6A shows a brain embryoid body embedded in a spherical Matrigel® hydrogel at the top Fluorinert™ FC-40 biocompatible oil in a pipette tip. FIG. 6B shows a brain embryoid body embedded in a spherical GrowDex® cellulose hydrogel at the top Fluorinert™ FC-40 biocompatible oil in a pipette tip. FIG. 6C shows a brain embryoid body embedded in a spherical Collagen I (rat) hydrogel at the top Fluorinert™ FC-40 biocompatible oil in a pipette tip. The use of the biocompatible oil results in EBs embedded in spherical hydrogels in each hydrogel tested.

### Example 4. Embryoid Body Embedding Using Standard 96 Well Plate and Advanced Pipetting Protocol

In this example, automated embryoid body embedding with oil is performed with immediate transfer to a bioreactor. The following automated steps are performed.
1. An embryoid body is added to media in the main well of a separation well microplate.
2. The plate is tilted to move the embryoid body far away from the separation channel (to the opposite side) so as to not lose the organoid during media removal.
3. The media is removed via the feeding well after repositioning the plate to a flat horizontal position.
4. The plate is tilted, and cold liquid Matrigel is added to the main well via pipette at the corner to encapsulate the embryoid body.
5. A biocompatible oil is added to the main well to reshape the hydrogel from a half moon shape to a more spheric shape.
6. The same pipette tip is positioned inside the Matrigel and used to slowly aspirate the Matrigel and oil.
7. The Matrigel will be then at the top of the tip and the oil due to the density in the lower part (FIG. 4, lower middle panel).
8. The embedded EB in Matrigel and the oil is transferred to an oil pool that was warmed up to 37° C., this pool had a stream that brings the embedded organoid to a bioreactor.
9. Once the Matrigel with the embryoid body (=future organoid) was solidified, the bioreactor containing a filter is emptied, washed once to remove possible left-over oil and the final media is added.
10. (Alternative to step 9) the organoids will go to a fluidic system where the embedded organoid in the oil steam will meet with normal media. Due to different densities, the oil will be removed through a filter system. Once the embedded organoid is situated in the complete media it will be redirected to the bioreactor.

### Clauses

Clause 1. A method for culturing of organoids, the method comprising
adding an embryoid body into a first media within a primary well of a separation well microplate comprising a plurality of well units, each well unit comprising a primary well, a secondary well, and one or more microchannels connecting the primary well to the secondary well;
removing the first media from the well unit via the secondary well;
adding a liquid hydrogel to encapsulate the embryoid body;
incubating the plate over a first period of time to solidify the hydrogel encapsulating the embryoid body; and
adding a second media to the secondary well to detach the solidified hydrogel encapsulating the embryoid body from the plate and suspend in the second media.

Clause 2. The method of clause 1, wherein the adding of the embryoid body into the first media within the primary well comprises automated pipetting.

Clause 3. The method of clause 1 or 2, further comprising
tilting the separation well microplate to move the embryoid body to a corner of the main well prior to removing the first media, optionally wherein the tilting comprises automated tilting of the separation well microplate.

Clause 4. The method of clause 3, wherein the tilting comprises moving the embryoid body toward the microchannels, optionally wherein the tilting comprises tilting the plate to an angle of from 1 to 60 degrees, 5 to 45 degrees, 5 to 35 degrees, 5 to 25 degrees, 5 to 15 degrees, 7 to 12 degrees, or about 10 degrees from a horizontal position.

Clause 5. The method of clause 3, wherein the tilting comprises moving the embryoid body away from the microchannels, optionally wherein the tilting comprises tilting the plate to an angle of from 1 to 60 degrees, 5 to 15 degrees, 7 to 12 degrees, or about 10 degrees from a horizontal position.

Clause 6. The method of any one of clauses 3 to 5, further comprising
returning the microplate to a horizontal position before removing the second media.

Clause 7. The method of any one of clauses 1 to 6, wherein the adding of the liquid hydrogel to encapsulate the embryoid body comprises image guided pipetting of the liquid hydrogel, optionally wherein the image guided pipetting is automated image guided pipetting comprising delivery of the liquid hydrogel on top of at least a portion of the embryoid body.

Clause 8. The method of any one of clauses 1 to 7, further comprising cooling the microplate before adding the liquid hydrogel, optionally wherein the cooling is automated cooling to a temperature below room temperature, or within a temperature range of 4 to 8 deg C.

Clause 9. The method of any one of clauses 1 to 7, further comprising heating the microplate to incubation temperature before adding the liquid hydrogel, optionally wherein the heating is automated heating.

Clause 10. The method of any one of clauses 1 to 9, comprising
further tilting the microplate to move the solidified hydrogel encapsulating the embryoid body away from the microchannels after adding the second media, optionally wherein the further tilting comprises automated further tilting.

Clause 11. The method of any one of clauses 1 to 10, wherein the incubating to solidify the hydrogel occurs over a first period of time in a range selected from the group consisting of from about 5 minutes to about 90 minutes, about 10 minutes to about 60 minutes, about 20 minutes to about 40 minutes, and about 30 minutes, optionally wherein the microplate is held at room temperature for a second period of time prior to the incubating step.

Clause 12. The method of any one of clauses 1 to 11, further comprising
exchanging the second media in the well unit via the secondary well periodically to feed the embryoid body.

Clause 13. The method of any one of clauses 1 to 12, further comprising
introducing a biocompatible oil into the primary well after adding the liquid hydrogel to encapsulate the embryoid body and before the incubating, optionally wherein the introducing comprises automated pipetting of the biocompatible oil into the primary well.

Clause 14. The method of clause 13, wherein the introducing comprises
positioning a pipette tip inside the hydrogel and aspirating the hydrogel encapsulating the embryoid body and the oil into the pipette tip, optionally wherein the positioning is image guided positioning.

Clause 15. The method of clause 14, wherein the aspirating is performed slowly over a period of at least 0.3 seconds, at least 0.5 seconds, at least 0.7 seconds, or at least 1 second.

Clause 16. The method of clause 14 or 15, further comprising
pushing the oil and the hydrogel encapsulating the embryoid body from the pipette tip back into the primary well.

Clause 17. The method of clause 16, wherein the pushing is performed slowly over a period of at least 0.3 seconds, at least 0.5 seconds, at least 0.7 seconds, or at least 1 second.

Clause 18. The method of any one of clauses 13 to 17, further comprising
withdrawing the biocompatible oil from the primary well via the secondary well after solidifying the hydrogel encapsulating the embryoid body; and
washing the well unit with a wash media prior to adding the second media, optionally wherein the wash media is at room temperature.

Clause 19. The method of any one of clauses 13 to 18, wherein the introducing of the biocompatible oil to the primary well causes the hydrogel encapsulating the embryoid body to become closer to a spherical shape.

Clause 20. The method of any one of clauses 1 to 19, further comprising
transferring the encapsulated embryoid body embedded in the solidified hydrogel to a bioreactor for maturation.

Clause 21. The method of any one of clauses 13 to 20, further comprising
transferring the hydrogel encapsulating the embryoid body and the biocompatible oil to an oil pool that is warmed to incubation temperature to solidify the hydrogel encapsulating the embryoid body.

Clause 22. The method of clause 21, wherein the oil pool comprises a stream that transfers the hydrogel encapsulating the embryoid body to a bioreactor optionally comprising a filter.

Clause 23. The method of clause 22, further comprising
emptying the oil from the bioreactor wherein the filter retains the solidified hydrogel encapsulating the embryoid body;
washing the bioreactor and retained solidified hydrogel encapsulating the embryoid body; and
adding a third media to the bioreactor to feed the washed hydrogel encapsulating the embryoid body.

Clause 24. The method of clause 22, wherein the oil pool stream that transfers the hydrogel encapsulating the embryoid body into the bioreactor meets with a third media; and the method further comprises
allowing the mixture of oil and third media to separate into layers; and
removing the oil layer through a filter system to retain the solidified hydrogel encapsulating the embryoid body in the third media; and
directing the solidified hydrogel encapsulating the embryoid body in the third media to the bioreactor.

Clause 25. The method of any one of clauses 1 to 24, wherein the method is an automated method, wherein the adding of the embryoid body to the first media, the removing of the first media from the well unit, the adding of the liquid hydrogel, and the adding of the second media each comprise automated pipetting.

Clause 26. The method of any one of clauses 1 to 25, wherein the embryoid body is a differentiated embryoid body exhibiting germ layer differentiation.

Clause 27. The method of any one of clauses 1 to 26, wherein the organoids are brain organoids.

Clause 28. The method of clause 27, wherein the brain organoids are cerebral organoids.

Clause 29. The method of any one of clauses 1 to 28, wherein the hydrogel is selected from the group consisting of a murine Engelbreth-Holm-Swarm (EHS) sarcoma matrix, a collagen type I, fibrin, hyaluronic acid (HA), gelatin methacrylate (GelMA), a decellularized matrix, alginate, silk, nanocellulose, polyethylene glycol (PEG), a self-assembling peptide, a poly (lactic/(co) glycolic) acid, a polycaprolactone, a polyacrylamide, oligo (ethylene glycol)-substituted poly isocyanopeptide, and an ELP (elastin-like protein).

Clause 30. The method of any one of clauses 1 to 29, wherein the liquid hydrogel is a cold liquid hydrogel, optionally at a temperature below about 10° C.

Clause 31. The method of any one of clauses 13 to 30, wherein the biocompatible oil has a density different than water.

Clause 32. The method of clause 31, wherein the biocompatible oil has a density higher than water.

Clause 33. The method of clause 32, wherein the biocompatible oil is a halogenated hydrocarbon, optionally a perfluorinated hydrocarbon.

Clause 34. The method of clause 31, wherein the biocompatible oil has a density less than water.

Clause 35. The method of clause 34, wherein the biocompatible oil is a silicone oil, a mineral oil, or a vegetable oil.

1. A method for culturing of organoids, the method comprising
adding an embryoid body into a first media within a primary well of a separation well microplate comprising a plurality of well units, each well unit comprising a primary well, a secondary well, and one or more microchannels connecting the primary well to the secondary well;
removing the first media from the well unit via the secondary well;
adding a liquid hydrogel to encapsulate the embryoid body;
incubating the separation well microplate over a first period of time to solidify the liquid hydrogel encapsulating the embryoid body into a solidified hydrogel; and
adding a second media to the secondary well to detach the solidified hydrogel encapsulating the embryoid body from the plate and suspend in the second media.

2. The method of claim 1, further comprising
tilting the separation well microplate to move the embryoid body to a corner of the main well prior to removing the first media.

3. The method of claim 2, wherein tilting the separation well microplate comprises moving the embryoid body away from the microchannels.

4. The method of claim 2, further comprising
returning the separation well microplate to a horizontal position before removing the second media.

5. The method of claim 1, wherein adding the liquid hydrogel to encapsulate the embryoid body comprises image guided pipetting of the liquid hydrogel.

6. The method of claim 1, further comprising cooling the separation well microplate before adding the liquid hydrogel.

7. The method of claim 1, further comprising heating the separation well microplate to incubation temperature before adding the liquid hydrogel.

8. The method of claim 1, further comprising tilting the separation well microplate to move the solidified hydrogel encapsulating the embryoid body away from the one or more microchannels after adding the second media.

9. The method claim 1, wherein the first period of time comprises a range selected from the group consisting of: about 5 minutes to about 90 minutes, about 10 minutes to about 60 minutes, about 20 minutes to about 40 minutes, and about 30 minutes.

10. The method of claim 1, further comprising
exchanging the second media in the well unit via the secondary well periodically to feed the embryoid body.

11. The method of claim 1, further comprising
introducing a biocompatible oil into the primary well after adding the liquid hydrogel to encapsulate the embryoid body and before incubating the separation well microplate.

12. The method of claim 11, wherein introducing the biocompatible oil into the primary well comprises positioning a pipette tip inside the hydrogel encapsulating the embryoid body and aspirating the hydrogel encapsulating the embryoid body and the biocompatible oil into the pipette tip.

13. The method of claim 12, further comprising
pushing the oil and the hydrogel encapsulating the embryoid body from the pipette tip back into the primary well.

14. The method of claim 11, further comprising
withdrawing the biocompatible oil from the primary well via the secondary well after solidifying the hydrogel encapsulating the embryoid body; and
washing the well unit with a wash media prior to adding the second media.

15. The method of claim 1, further comprising
transferring the encapsulated embryoid body embedded in the solidified hydrogel to a bioreactor for maturation.

16. The method of claim 11, further comprising
transferring the liquid hydrogel encapsulating the embryoid body and the biocompatible oil to an oil pool that is warmed to incubation temperature to solidify the liquid hydrogel encapsulating the embryoid body into the solidified hydrogel, wherein the oil pool comprises a stream that transfers the hydrogel encapsulating the embryoid body to a bioreactor comprising a filter.

17. The method of claim 16, further comprising
emptying the oil from the bioreactor wherein the filter retains the solidified hydrogel encapsulating the embryoid body;
washing the bioreactor and retained solidified hydrogel encapsulating the embryoid body; and
adding a third media to the bioreactor to feed the washed hydrogel encapsulating the embryoid body.

18. The method of claim 16, wherein the stream that transfers the hydrogel encapsulating the embryoid body to the bioreactor meets with a third media; and the method further comprises:
allowing a mixture of the biocompatible oil and the third media to separate into an oil layer and a layer of the third media; and
removing the oil layer through a filter system to retain the solidified hydrogel encapsulating the embryoid body in the third media; and
directing the solidified hydrogel encapsulating the embryoid body in the third media to the bioreactor.

19. The method of claim 1, wherein the method is an automated method, and wherein adding the embryoid body into the first media, removing the first media from the well unit, adding the liquid hydrogel, and adding the second media each comprise automated pipetting.

20. The method of claim 1, wherein the embryoid body is a differentiated embryoid body exhibiting germ layer differentiation.

21. The method of claim 1, wherein the liquid hydrogel is selected from the group consisting of: a murine Engelbreth-Holm-Swarm (EHS) sarcoma matrix, a collagen type I, fibrin, hyaluronic acid (HA), gelatin methacrylate (GelMA), a decellularized matrix, alginate, silk, nanocellulose, polyethylene glycol (PEG), a self-assembling peptide, a poly (lactic/(co)glycolic) acid, a polycaprolactone, a polyacrylamide, oligo (ethylene glycol)-substituted poly isocyanopeptide, and an ELP (elastin-like protein).

22. The method of claim 11, wherein the biocompatible oil has a density that is different than a density of water.

23. The method of claim 22, wherein the biocompatible oil has a density that is higher than the density of water.

24. The method of claim 22, wherein the biocompatible oil has a density that is less than the density of water.
